(19) United States
(12) Patent Application Publication
(10) Pub. No.: US 2008/0311093 A1
(43) Pub. Date: Dec. 18, 2008

(54) STEM CELL SECRETIONS AND RELATED METHODS

(75) Inventor: Keith K. Skinner, Denver, CO (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: AMERICAN SYMBOLIC, LLC

(21) Appl. No.: 11/952,898

(22) Filed: Dec. 7, 2007

Related U.S. Application Data
(60) Provisional application No. 60/868,971, filed on Dec. 7, 2006.
(60) Provisional application No. 60/945,014, filed on Jun. 19, 2007.
(60) Provisional application No. 60/952,535, filed on Jul. 27, 2007.

(51) Int. Cl. A61K 35/12; A61Q 99/00

(52) U.S. Cl. 424/93.21; 424/93.7

(57) ABSTRACT

Stem cell secretions are derived from epithelial cells conditioned media. The stem cell secretions are then applied topically, orally, or rectally, etc., to derive health benefits from the growth factors and other molecules comprising the stem cell secretion. The stem cell secretion may optionally be modified by covalently bonding fatty acids to protect the molecules through the delivery process and to make them more readily available to cells.

### RELATED APPLICATION

This application claims the benefit of and Paris Convention Priority to U.S. Provisional Application Ser. No. 60/868,971, filed Dec. 7, 2006; and U.S. Provisional Application Ser. No. 60/945,014, filed Jun. 19, 2007; and U.S. Provisional Application Ser. No. 60/952,535, filed Jul. 27, 2007; and the contents of each is hereby incorporated by reference herein in its entirety.

### BACKGROUND

The present disclosure relates to the field of stem cell conditioned media and more particularly to stem cell secretions, lyophilization, and stabilization of the secretions. The constituents of the stem cell secretion may be protected with fatty acids to both prevent premature breakdown and improve cellular uptake when used as an epidermal stem cell secretion composition for topical and systemic administration, the compounding of the stem cell secretion composition to provide cosmetic formulations suitable for therapeutic use and test methods for determining the bio-active effectiveness of the stem cell secretion.

### SUMMARY

Stem cell secretions are derived from epithelial stem cell conditioned media. The stem cell secretions are then applied topically, orally, or rectally, etc., to derive health benefits from the growth factors and other molecules comprising the stem cell secretion. The stem cell secretion may optionally be modified by covalently bonding fatty acids to protect the molecules through the delivery process and to make them more readily available to cells.

According to a feature of the present disclosure, a method is disclosed comprising harvesting stem cells, culturing the stem cells, separating the a stem cell conditioned media from the stem cells and other debris to produce purified stem cell secretions, and providing the stem cell secretions to be delivered to an animal.

According to a feature of the present disclosure, a method is comprising harvesting stem cells, culturing the stem cells, removing a stem cell conditioned media having stem cell secretions, adding a fatty acid chloride source to the aqueous solution at pH 11-14, stirring, purifying the resulting precipitate comprising at least fatty acid modified stem cell secretions.

According to features of the present disclosure, products by the above processes are also disclosed.

According to a feature of the present disclosure, a composition is disclosed comprising stem cell secretions harvested from a conditioned stem cell media and separated from the stem cells from which the stem cell secretions are derived, wherein the stem cell secretions have at least one fatty acid chemically attached thereto.

### DRAWINGS

The above-mentioned features and objects of the present disclosure will become more apparent with reference to the following description taken in conjunction with the accompanying drawings wherein like reference numerals denote like elements and in which:

FIG. 1 is a flow diagram of an embodiment of a method for creating a stem cell secretions additive;

FIG. 2 is a flow diagram illustrating an embodiment of a process for creating stem cell secretions; and

FIG. 3 is a flow diagram illustrating an embodiment of a process for modifying stem cell secretions with fatty acids.

### DETAILED DESCRIPTION

These embodiments are described in sufficient detail to enable those skilled in the art to practice the invention, and it is to be understood that other embodiments may be utilized and that logical, mechanical, electrical, biological, functional, and other changes may be made without departing from the scope of the present invention. The following detailed description is, therefore, not to be taken in a limiting sense, and the scope of the present invention is defined only by the appended claims. As used in the present disclosure, the term “or” shall be understood to be defined as a logical disjunction and shall not indicate an exclusive disjunction unless expressly indicated as such or notated as “xor.”

As used in the present disclosure, the term “stem cell additive” or “stem cell secretion(s)” shall be understood to mean secretions of stem cells or non-stem cells grown in the same media with the stem cells. The secretions expressly include growth factors, regulatory proteins, hormones, signaling proteins, or other organic molecules secreted from the stem cells or non-stem cells grown in the same media as the stem cells.

Stem cells are primal undifferentiated cells, present in small numbers among differentiated cells in tissues and organs. Stem cells have the ability to differentiate into different cell types to act as a mechanism of repair for damaged tissues. They secrete various signaling factors such as proteins that have the ability to maintain and renew aging tissues.

There are two types of stem cells: those found in adult tissues (“Somatic Stem Cells”), which are “multipotent,” because they multiply themselves into cells of a specific type or family of cells; and embryonic stem cells, which are able to give rise to all the cell types seen in an adult organism, and are therefore said to be “totipotent.” In humans, specialized cells cannot backtrack and start over. Generally, the most cells are able do is to return from a non-dividing state to a dividing one, as for example connective tissue and bone cells do in healing a wound or break, and as skin cells do in regenerating skin lost through burns or cuts. It is believed that nerve cells can regrow their axons and dendrites, but nerve cells themselves cannot be replaced. However, some adult cell types can dedifferentiate slightly, for example, as some connective tissue cells do to produce new bone cells. Generally, more extensive differentiation is considered abnormal and is a diagnostic sign of certain cancers. Adult hair follicles provide an example of normal healthy crossover between different organ adult stem cells, as they can occasionally act as bone or fat stem cells, and may differentiate into bone or fat.

However, many adult tissues possess a “reservoir” of stem cells that demonstrate a high degree of plasticity. Researchers are investigating adult stem cells from a variety of sources, including, skin/hair follicles, adipose (fatty) tissue, the synovial membrane (inner layer of lubricating capsule surrounding free moving joints), perivascular cells (related to or occurring in tissues surrounding blood vessels), and tooth dental pulp cells. The adult stem cells isolated from the skin (“dermis”) were shown to be able to differentiate in laboratory culture dishes giving rise to muscle and neurons. However, the observable characteristics produced by the interaction of genetic make up and environmental factors, as well as the origin of this stem cell population is unclear, as the initial dermal population was heterogeneous (no uniform composition).

Hair follicle epithelial cells have been the subject of intense study in relation to skin renewal and tumor biology. As seen during the course of normal follicle growth and cycling, dynamic epithelial (membrane linings of cavities or tubes)-mesenchymal (loosely organized undifferentiated mesodermal, or middle layer, cells that give rise to such structures as connective tissues, blood, lymphatics, bone, and cartilage) persists from embryonic development into adulthood.

Hair follicles contain discrete populations of interacting cells that are clustered in defined sites and can be isolated, cultured, and then experimentally manipulated. Thus, the hair follicle is emerging as a major developmental stem-cell model, encompassing paradigms of epithelial-mesenchymal interactions and epithelial stem cell behavior, as well as high accessibility in the adult body. The follicle dermis (skin) acts as an important stem cell repository for repair of the dermis after skin wounding. Moreover, the stem cell hematopoietic (blood stream) activity was demonstrated in the follicle dermis. In a study examining the capacity of rat follicles from the hair follicle dermal papilla (DP) and dermal sheath (DS), to differentiate into adipogenic (fatty cells) or osteogenic (bone cell), it was concluded that cells from the hair follicle DP and DS can differentiate either spontaneously or in direct fashion along other mesodermal lineages in vitro.

The skin is currently one of the few organs in which adult stem cells can be maintained and propagated in the laboratory. With respect to the epidermis, cells are generated through proliferation that occurs only in the basal layer; therefore, stem cells must be located there. Within the basal layer, keratinocytes display such heterogeneous proliferative characteristics. Within the epidermis, the main source of the stem cells responsible for continual epidermal renewal appears to reside in the center of the EPU (epidermal proliferative unit). Similar to this concept, the hair follicle generates a terminally differentiated keratinized end product, the hair shaft that is eventually shed.

Keratinocytes within the budge area may depend on their environment or niche for maintaining their stem cell characteristics. The bulge area also houses melanocyte stem cells, which are normally quiescent but proliferate at anagen onset to repopulate the new lower anagen hair follicle with melanocytes that generate melanin leading to pigmentation of their hair.

Under normal homeostatic conditions, epidermal stem cells and hair follicle stem cells constitute two distinct populations and understanding of differences in their structures and means of communication with other cells is of importance for developing treatments based on stem cells.

The development of the hair follicles represents a series of interactions between cells derived from embryonic ectoderm and mesoderm. Both an increase and a decrease in the expression of various growth factors, adhesion molecules, glycosaminoglycans, etc., has been seen during this development process. Yet, their roles in different stages of hair cycle have not been identified. It is generally assumed that follicular development and hair cycling is a consequence of messages passing between the epidermal compartment of the follicle and the dermal compartment. The epidermal compartment was shown in animal experiments to play a role in follicle development, the fate of the emerging hair structure, and provide signals for a new round of hair growth or maintenance of the anagen phase. These epithelium-derived signals presumably affect papilla development and possibly growth of the surrounding epithelial matrix compartments. Previous research demonstrated growth properties of human scalp-derived dermal papilla (hDP) cells. In addition, human DP cells failed to proliferate out of intact papilla when maintained in a growth medium specifically designed for human keratinocyte growth, (keratinocyte growth medium KGM). Human scalp-derived papilla (hDP) cells grew when plated together with human epithelial keratinocytes (hK), which suggests that hK support growth either directly (transmission of contact-dependent growth signals) or indirectly (transmission of diffusible growth signals). Establishment of a physical barrier between hK and hDP cells had no effect on hK's ability of transmitting growth signals. The stimulation of radiolabeled thymidine incorporation into quiescent hDP cells has shown that the medium conditioned by hK contained mitogenic factors for hDP cells since it induced a 2-5-fold increase in mitogenic activity in hDP. The mitogenic activity of hK conditioned medium appeared to be specific to fibroblast-like cells.

The skin healant properties of the conditioned medium of cultured human keratinocytes (KCM) containing the autocrine and paracrine activities of growth factors they produce was shown by the ability of cultured human keratinocytes (KCM) to display growth stimulating properties on several cell lines involved in wound healing process as well as the ability of KCM to stimulate re-epithalization of wounds in human skin.

Collagenases, the enzymes that degrade collagen, regulate such process in wound healing as the migration of keratinocytes over the wound bed, angiogenesis, and remodeling of the granulation tissue. Degradation of collagen is necessary for the wound healing process. To investigate the autocrine and paracrine control of collagenase production in human keratinocytes, cultured human keratinocytes and fibroblasts were stimulated with KCM and assessed for collagenase production by binding studies with tritiated diisopropylfluorophosphate (3H-DFP) and zymography. Lysates from unstimaluted human keratinocytes contained 3H-DFP binding proteins of both 72 and 92 kDa. Cells exposed to KCM showed the same 72 and 92 kDa bands as unstimulated cells, and showed a 49% enhancement of the band at 72 kDa and a 19% enhancement of the band at 92 kDa. Human fibroblasts were also shown to have increased production of 72 kDa collagenase after KCM stimulation. Thus, the study showed that conditioned medium from keratinocytes up-regulates the expression of 72 and 92 kDa type IV collagenases in human keratinocytes, and the 72 kDa collagenase in human fibroblasts, indicating the presence of an autocrine/paracrine control mechanism, which regulates the collagenase production in these cell types. By production of autocrine and paracrine factors, keratinocytes may play an important part in the control of several events involved in wound healing, such as production and remodeling of granulation tissue, and re-epithelialization of the wound.

Stem cells have been shown to produce soluble factors, known and unknown, found in “conditioned media.” Conditioned media is a soup produced by the secretory output of growing epithelial stem cells in culture; this soup is known to facilitate normal growth of skin and hair follicle cell types.

In an investigation of the ability of the skin from the body of a patient exposed to intensive injury to produce Hepatocyte Growth Factor (HGF) as well as the production, properties and effects of HGF in conditioned medium, samples taken from injured skin on the legs of a flame-burnt male patient and keratinocyte monolayer were obtained from the sample (injured keratinocytes). The medium was collected every fifth day, centrifuged and stored. The conditioned medium from a culture of human injured keratinocytes was centrifuged and the supernatant was used for studies. To investigate whether HGF production by skin keratinocytes was limited to cases with major trauma, normal skin keratinocytes were cultured under the same conditions, immuno-chemical staining of deeply injured skin revealed that some vessels were layered with endothelial cells that highly expressed HGF, while HGF expression in endothelial and epithelial cells of normal skin from healthy volunteers was low. Western blot analysis of the conditioned medium from cultured skin cells (injured keratinocytes) showed that the cells produced human keratin. Analysis of the medium obtained from injured keratinocytes detected HGF affinity for anti-HGF antibodies, the HGF receptor, human albumin, and dextran sulfate.

The extracellular matrix (ECM) is made of a wide variety of components with predominance of collagens and noncollagenous proteins. These two groups of compounds contribute to the maintenance of tissue integrity and architecture. In addition, they affect cell behavior. Dermatopontin is one of the noncollagenous components of the ECM. In humans, dermatopontin is detected in fibroblasts, in the heart, muscle, and lungs. In skin, dermatopontin tends to be distributed around collagen fibers and within endothelial cells. Dermatopontin plays a vital role in ECM architecture (interaction with decorin and modification of collagen fibrillogenesis), cell behavior (a weak adhesion activity for certain fibroblasts and neurogenic cells; interaction with TGF-beta bioactivity and modification of collagen fibrillogenesis, possibly by interacting with collagen molecules), and pathological involvements (increased expression around myocardial infarct zone, decreased expression in leimyoma and keloid, and decreased expression in fibrosing diseases). In addition, a change in the dermatopontin expression also has been reported in the chondrocyte culture system. Scientists suggest that dermatopontin could interact with type I collagen and modify its fibrillogenesis thus aiding at maintaining the mechanical strength or elasticity of the cartilage.

Experiments have shown that fibroblasts are capable of producing stem cell factors (SCF) and mast cell differentiation factors. SCF is a multifactorial regulator of hematopoietic stem cell, mast cell, and melanocyte differentiation and function. Because keratinocytes are capable of producing SCF, the inventors investigated whether keratinocytes can produce mast cell differentiation. The results have shown that, using human HaCaT keratinocyte cell lines, the keratinocyte supernatants produce and release factors that upregulate mast cell characteristics. It was shown that differentiating keratinocytes displayed much higher ability to release these factors than proliferating HaCaT keratinocytes. This activity is not likely to be due to SCF since human mast cell line HMC-1 cells have been shown to be poor responders to SCF. The inventors concluded that keratinocytes release both SCF and differentiation-dependent factors which regulate mast cell development.

Moreover, different cell types communicate among themselves through a variety of signals. It is hypothesized that the production of these signals is connected to soluble factors with autocrine and paracrine activities; cell-matrix; and cell-cell interaction. Cultured epidermis contains both keratinocytes and melanocytes. Some evidence suggests that epidermal keratinocytes secrete soluble proteins responsible for modulation of the growth of keratinocytes, melanocytes, and other cells outside the epidermis. Thus, inventors investigated whether soluble factors derived from normal human epidermal keratinocytes are capable to show a paracrine effect on both melanocytes and dermal fibroblasts by influencing their spread, density, and cell-cell contact. With the increased cell density of the epidermal cell population, the percentage of melanocytes involved in cell-cell contacts increases but eventually plateaus. The lower fraction of keratinocytes involved in keratinocyte/keratinocyte contacts than that of melanocytes suggests a chemosatic mechanisms preferentially acting on melanocytes rather than the keratinocytes themselves. Keratinocyte-induced dendricity of cultured melanocytes is triggered by the early secretion of at least one unidentified factor(s) by keratinocytes. Approximately one-half of factors showing growth promoting activity from conditioned medium, as well as most of the dendricity and melanization stimulating activities were of low (less than 500 Daltons) molecular weight.

Skin adsorption of molecules rapidly declines as their size increases above 500 Daltons due to the corneal layer. Where the corneal layer is absent, such as in mucous membranes, larger molecules more efficiently adsorb. For pharmaceutical development purposes, it seems logical to restrict the development of new innovative compounds to a molecular weight under 500 Dalton, when topical therapy is objective.

The culture of hair cell follicle derived stem cells has been performed by the methods disclosed in U.S. Pat. No. 5,556,783, which is incorporated by reference herein. The stimulation of their growth has been performed by several different methods, including the teachings of U.S. Pat. No. 5,902,741, which is likewise incorporated by reference, who utilize a three-dimensional culture medium and TGF-beta. U.S. Pat. Nos. 5,962,325, and 6,022,743, which are incorporated by reference, teaches the use of a similar methods for the culture of mesenchymal cells and pancreatic parenchymal cells. Extracellular collagen matrices, as disclosed in U.S. Patent Application No. 2005/0106723, which is incorporated by reference, have also been used. Other inventors have isolated a specific polypeptide from skin cell culture that stimulates epithelial cell growth in U.S. Patent Application No. 2003/0040471, which is also incorporated by reference.

The culture of hair cell follicle derived stem cells has been performed by the method of Lakver, et al, U.S. Pat. No. 5,556,783. The stimulation of their growth has been performed by several different methods, including the teachings of U.S. Pat. No. 5,902,741, to Purchio, et al, who utilize a three-dimensional culture medium and TGF-beta. Naughton, U.S. Pat. No. 5,962,325, teaches the use of a similar method for the culture of mesenchymal cells and pancreatic parenchymal cells, U.S. Pat. No. 6,022,743. Extacellular collagen matrices, U.S. Patent Application No. 20050106723, to Hatzfeld, have also been used. Other inventors have isolated a specific polypeptide from skin cell culture that stimulates epithelial cell growth, U.S. Patent Application No. 20030040471, to Watson.

The inventors have discovered that the stimulation of a stem cell culture, specifically cells derived from the bulge area of a hair follicle, results in substantial increase in both the expansion of the cell population and its release of signaling factors, growth factors, and other biological molecules into the conditioned growth media. Moreover, the inventors have discovered that the stem cell secretions derived according to the instant teachings may be modified by bonding fatty acids to their active sites, thereby protecting the stem cell secretions and creating a vehicle for more efficient delivery intracellularly.

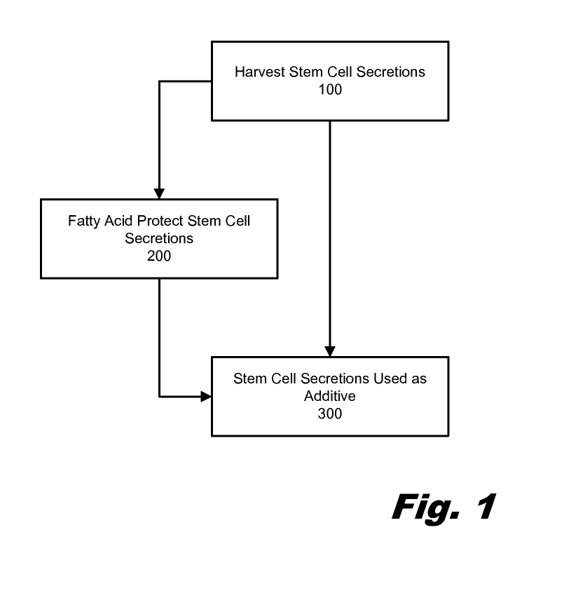

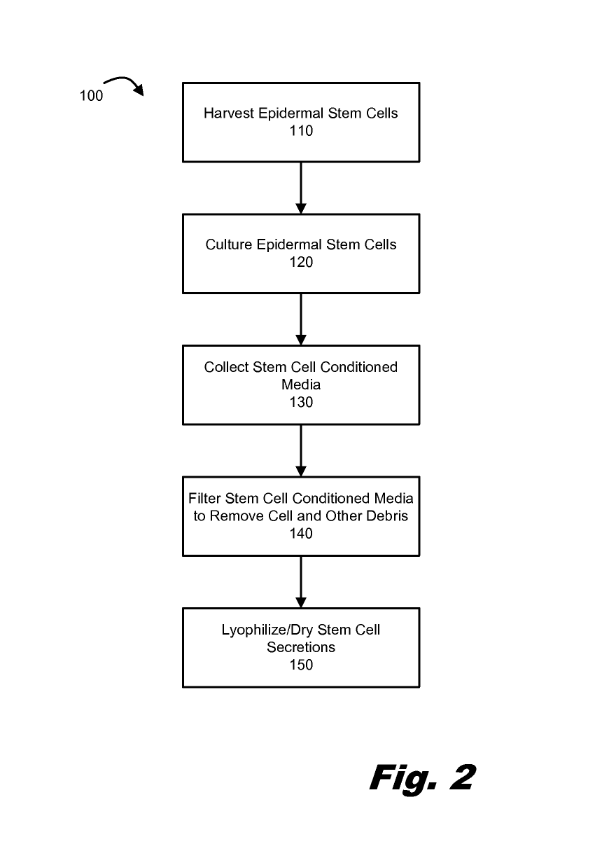

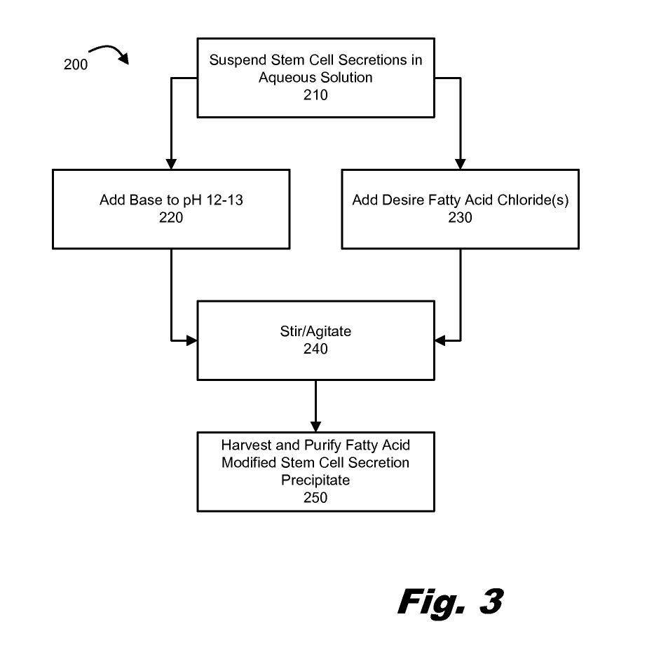

As illustrated in FIG. 1, which illustrates an embodiment an overview of the processes of the present disclosure, stem cell secretions are harvested in operation 100, which is shown in greater detail in FIG. 2. The stem cell secretions may then be optionally modified with fatty acids in operation 200, which is illustrated in greater detail in FIG. 3. The resulting stem cell secretions or fatty acid modified stem cell secretions may be used, for example, as additives in a variety of products, from lotions and skin creams to orally consumed therapeutic products to cooking oils in operation 300.

According to an exemplary embodiment of a method of making an epidermal stem cell secretions, and as illustrated in FIG. 2, one or more hair follicles comprising at least one actively growing epidermal stem cell are extracted and the at least one hair follicle is enzymatically treated to separate them from the one or more hair follicles in operation 110. In operation 120, the one or more epidermal stem cells are cultured to provide a first population of stem cells and the first population of stem cells is purified by methods well understood in the art. According to the embodiments, the first population of stem cells is then cultured to provide a second population of stem cells, as is well understood in the art. According to the present disclosure, the second population of stem cells may be transferred to a second medium to culture the second population of stem cells. Without limiting the disclosure and merely to illustrate the type of medium described herein, in an aspect the medium may comprise Eagle's serum free alpha-MEM, 10-8 M dexamethasone, 10 mM sodium beta-glycerophosphate, and 50 mg/ml L-ascorbic acid 2-phosphate. In a further step the second population of stem cells may be stimulated with at least one low intensity pulsed ultrasound source to provide a third population of stem cells and a conditioned medium having stem cell secretions (the first and second mediums also have stem cell secretions that can be harvested). The third population of stem cells may be separated from the conditioned medium in operation 130, and the conditioned medium may be filtered to remove cells and debris in operation 140. Artisans will readily appreciate that most, if not all, cell culture protocols may be used to derive the stem cell secretions of the present disclosure. Furthermore and according to embodiments, the conditioned medium may be filtered to sterilize the medium, and the stem cell secretions may be freeze-dried and lyophilized to provide one or more epidermal stem cell additives in operation 150.

It will be appreciated that the conditioned media having stem cell secretions made according to the above disclosed method comprises a “conditioned soup” produced by the secretory output of growing skin stem cells in a culture medium. According to embodiments, the skin stem cells may comprise material obtained from a bulge location of one or more hair follicles. Without limiting the disclosure, the resulting “conditioned soup” may comprise a variety of factors which may facilitate therapeutic treatment of the epidermal structure of a skin tissue. Such factors include, but are not limited to: hepatocyte growth factor (HGF), dermatopontin, and other stem cell growth factors and the like. In one aspect, the epidermal stem cell additive may comprise a powder form, although it will be appreciated that the additive may be formulated in any suitable manner as is understood in the art.

According to embodiments, the first population of stem cells may be obtained in a yield of about 10 to about 2000 cells during culturing. Furthermore, in another aspect the first population of stem cells may be obtained in a yield of about 50 to about 1000 cells. In yet another embodiment the first population of stem cells may be obtained in a yield of about 100 to about 200 cells.

According to embodiments with respect to the second population of stem cells, these cells may be obtained in a yield of about 25,000 to about 2,500,000 cells. Alternatively, in another embodiment the second population of stem cells may have a yield of about 125,000 to about 1,250,000 cells. Furthermore, in yet another aspect of the disclosure the second population of stem cells may have a yield of about 250,000 cells.

According to embodiments the third population of stem cells may be expanded to a range between about 400,000 and about 40,000,000 stem cells. Furthermore, in another aspect the third population of stem cells may be expanded to a range between about 800,000 to about 20,000,000 stem cells. Alternatively, in yet another embodiment the third population of stem cells may expanded to about 4,000,000 stem cells during culturing.

According to an embodiment of the disclosure, the low intensity pulsed ultrasound source may have a signal speed ranging from about 0.15 MHz to about 15 MHz, an intensity of about 7 mW/cm² to about 700 mW/cm² and the source may be applied for about 2 minutes to about 400 minutes during each 24 hour interval of an about 24 hour to about 720 hour time period. In yet another embodiment, the low intensity pulsed ultrasound source may have a signal speed ranging from about 0.30 MHz to about 7.5 MHz, an intensity of about 14 mW/cm² to about 350 mW/cm² and the source may be applied for about 4 minutes to about 200 minutes per 24 hours of an about 24 hour to about 360 hour time period. In a further embodiment, the low intensity pulsed ultrasound source may have a signal speed of about 1.5 MHz, an intensity of about 70 mW/cm², and the source may be applied for about 20 minutes to about 40 minutes during each 24 hour interval of a time period of about 72 hours. The constituents of the resulting stem cell secretions may be modified by complexing with fatty acids as described herein. In an aspect of the method as described above, the stem cell secretions may be filtered to remove cells and debris and may comprise a filter having a pore size of about 2 microns to about 10 microns. In another aspect the filter may have a pore size of about 5 microns.

In another aspect in order to sterilize the stem cell secretions, a filter having a pore size of about 2 microns to about 0.02 microns may be used. In yet another embodiment the sterilizing filter may be about 0.2 microns in pore size.

During processing, in an exemplary embodiment the stem cell secretions may be stabilized with at least one of ethylenediaminetetraacetic acid (EDTA) and ethylene glycol bis(2-aminoethylether)-N,N,N′N′-tetraacetic acid (EGTA) or any other stabilizer as is known in the art.

The present disclosure also discloses a method for modifying the constituents of the stem cell secretions with lipids thereby making the constituents of the stem cell secretions more readily bioavailable. Moreover, adding fatty acids to constituents of the stem cell secretions further allows the skin to more easily and efficiently absorb the constituents of the stem cell secretions. In effect, adding fatty acids to constituents of the stem cell secretions creates both a vehicle for delivery through lipid bilayers of cells and the skin, and a “time release” effect as the constituents of the stem cell secretions are not bioavailable until the lipid side chains of the modified constituents of the stem cell secretions are cleaved. Fatty acids are enzymatically cleaved carbon by carbon. Thus, constituents of the stem cell secretions having longer fatty acids chains therefore take longer to become bioavailable than those having shorter fatty acid chains.

The present disclosure proposes a novel method of making constituents of the stem cell secretions more deliverable to cells by adding fatty acids to active sites on the constituents of the stem cell stem cell secretions. The fatty acids are covalently bonded to one or more active sites of the constituents of the stem cell secretions, thereby preventing the constituents of the stem cell secretions from reducing free radicals prior to delivery of the constituents of the stem cell secretions at the target location.

Artisans will recognize the constituents of the stem cell secretions that may benefit from the fatty acid protection methods of the present disclosure. Constituents of the stem cell secretions will have at least one active site that can reversibly react with the carboxyl end of fatty acids; these may include NH₂, SH₂, and OH sites, and others as will be known and understood by artisans. Indeed, these sites are preferentially bound in the following order: amino or any free binding site, then sulphydryl or any free binding site, and finally hydroxyl sites. As will readily be recognized by artisans, NH₂ sites are will be modified first due their positive charge.

However, modification of the hydroxyl active sites is advantageous because ether bonds form between the fatty acid and the constituents of the stem cell secretions. The ether bonds are more stable in biologic systems, which means that the cell takes longer to break down the fatty acid and expose the active site.

According to embodiments, constituents of the stem cell secretions that may be modified according to the present disclosure include, but are not limited to, constituents of the stem cell cultured media such as proteins, antioxidants (for example glutathione, hyaluronic acid, carnosine, and others), and other molecules having at least one active site that can be modified by covalent bonding of a fatty acid.

As well known to artisans, fatty acids comprise an aliphatic chain coupled to a carboxylic acid. According to embodiments, the carboxy end of fatty acids are reacted to the active sites of the constituents of the stem cell secretions. The fatty acid-stem cell secretions constituents complex serves two purposes. First, the fatty acids reversibly block the active sites of the constituents of the stem cell secretions until the constituents of the stem cell secretions are delivered intracellularly. Second, the lipophilic aliphatic side chain or chains of the fatty acids allow the constituents of the stem cell secretions to more readily cross the cell membrane and penetrate skin, for example. Thus, by coupling constituents of the stem cell secretions and fatty acids, a more potent and effective cellular delivery vehicle for the constituents of the stem cell secretions results.

According to embodiments, any fatty acid having 2 or more carbons in the aliphatic chain are suitable to be coupled to constituents of the stem cell secretions. The fatty acids may be saturated or unsaturated. According to embodiments, butanoic acid (C4:0), pantanoic acid (C5:0), hexanoic acid (C6:0), octanoic acid (C8:0), nananoic acid (C9:0), decanoic acid (C10:0), dodecanoic acid (C12:0), tetradecanoic acid (C14:0), hexadecanoic acid (C16:0), heptadecanoic acid (C17:0), octadecanoic acid (C18:0), icosanoic acid (C20:0), docosanoic acid (C22:0), tetracosanoic acid (C24:0), hexacosanoic acid (C26:0), heptacosanoic acid (C27:0), octasonoic acid (C28:0), triacontanoic acid (C30:0), dotriacontanoic acid (C30:0), dotriacontanoic acid (C32:0), dotriacontanoic acid (C32:0), tritriacontanoic acid (C33:0), tetratriacontanoic acid (C34:0), or pentatriacontanoic acid (C35:0) are saturated fatty acids that are readily available and that are appropriate for use with the present disclosure depending on the application. Fatty acids having more than 35 carbons and fatty acids having aliphatic chains of both an even and an odd number of carbons are equally applicable with the teachings of the present disclosure.

Similarly, unsaturated fatty acids having any number of double or triple bonds in both -cis or -trans configurations are expressly contemplated. For example, myristoleic acid (C14:1), palmitoleic acid (C16:1), oleic acid (C18:1), linoleic acid (C18:2), a-linoleic acid (C18:3), arachidonic acid (C20:4), eicosapentaenoic acid (C20:4), eicosapentaenoic acid (C20:5), erucic Acid (C22:1), or docosahexaenoic acid (C22:6) are examples of common unsaturated fatty acids that may be coupled to constituents of the stem cell secretions according to the present disclosure. Other unsaturated fatty acids are expressly contemplated, as would be known to artisans.

Moreover, according to embodiments, the fatty acids of the present disclosure may be oils, such as olive oil, jojoba oil, sunflower oil, safflower oil, rapeseed oil, corn oil, soya oil, wheat germ oil, cottonseed oil, almond oil or oils of other nuts, palm oil, coconut oil, vegetable oil, butter, lard, as well as other oils comprising, at least in part, fatty acids. Obviously, where the constituents of the stem cell secretions are to be delivered intracellularly, the oil or fatty acid must be non-toxic.

According to embodiments, the oil selected my comprise oils known to be nutritionally healthy, such as olive oil or omega-3 fatty acids. Use of such health-type oils may be of interest to the health food markets, etc. Moreover, according to embodiments the stem cell secretions may comprise or have added it to health food supplements, the total product of which may then to be sold as such or may be included with other additives, such as antioxidants, zinc oxides, or titanium dioxides, in skin creams or other cosmetic applications, for example. Other examples include cooking or dipping oils for oral consumption having the fatty acid modified stem cell secretions.

Once delivered intracellularly, enzymes within the cell cleave off the fatty acids, allowing the bioactive sites of the constituents of the stem cell secretions to become available. Cleaving of the fatty acids occurs carbon by carbon. Consequently, the longer the aliphatic chain of the fatty acid, the longer the constituents of the stem cell secretions will be protected by the fatty acid(s). Indeed, by using fatty acids/oils having different size aliphatic chains, a time release-like product is created whereby the constituents of the stem cell secretions having the shorter aliphatic chains become bioavailable more quickly on average than those having longer aliphatic chains.

As illustrated in FIG. 3, the process for protecting constituents of the stem cell secretions with fatty acids is performed in an aqueous solution using the fatty acid chloride of the fatty acids being used to modify the active sites of the constituents of the stem cell secretions in operation 210. As will be seen, it may be performed in quantities of scale without appreciable modification in the core steps of the procedure. The concentration of the stem cell secretions dissolved into aqueous solution may be as high as possible, according to embodiments. According to other embodiments, a more dilute concentration may be used to reduce steric hindrance for complete fatty acid coupling to large constituents.

To covalently bind the fatty acid to the constituents of the stem cell secretions, the pH is raised to pH 11-14 with a base in operation 220. According to embodiments, the base is an inorganic base, such as NaOH, which prevents undesirable side reactions. Throughout the modification process, the pH is kept in the range of pH 11-14 to drive the modification reaction. After the pH is raised to the requisite level, for example pH 12-13, the fatty acid chloride is added to drop-wise to the solution in operation 230 under agitation/stirring in operation 240, together with additional base to maintain the desired pH. As the fatty acid chloride is added to the active site(s) of each constituent of the stem cell secretions, the resulting product falls out of solution as a precipitate. According to similar embodiments, the solution need not have the pH raised before adding the fatty acid chloride and the base, whereby the pH will be raised as a matter of course during the reaction.

The precipitate, comprising the fatty acid modified stem cell secretions, is then harvested in operation 250. Harvesting may occur simply by decanting the water, washing the precipitate with water at least once, and drying. The resultant dry precipitate comprises the constituents of the stem cell secretions coupled to one or more fatty acid molecules. The precipitate may then be added as an additive to other products such as vitamin tablets, lotion, skin creams, etc., for delivery purposes as described subsequently. According to embodiments, nearly any product having the fatty acid modified stem cell secretions by the disclosed process are expressly contemplated.

It will be understood by artisans that the methods of the instant disclosure may be performed on a large scale without appreciable changes to the principles disclosed by the exemplary protocol.

According to embodiments, the fatty acid modified stem cell secretions constituent complex may be further modified, either before or after the process disclosed herein to provide further desirable characteristics. For example, antioxidant molecules, such as glutathione, may be esterified prior to the process disclosed herein. Other similar modifications that are known in the art, such as acetylation with glutathione, are also possible and expressly contemplated, provided active sites on the constituents of the stem cell secretions are available for modification.

### EXAMPLE 1

One gram of stem cell secretions (conditioned media) from CK-15/CK-19 adult human skin stem cells is dissolved into 500 mL of lab grade water (filtered to 0.2 micron with a milipore filter). The mixture is stirred with a votex stirrer at 3000 RPM to ensure complete resuspension of the stem cell secretions for 2 minutes.

The solution is then made basic to pH 12-13 by adding NaOH to the solution dropwise, which acylates the amine groups. Thereafter, a fatty acid chloride, for example palmitoyl chloride 98% (Fisher Scientific) is titrated into the solution together with NaOH to maintain the pH at 12-13. The modified stem cell secretions will precipitate out of solution as they are modified with the fatty acid molecules. HPLC may be used to determined purity, as known and understood by artisans.

### EXAMPLE 2

Similarly, the procedure of EXAMPLE 1 is duplicated. However, rather than using palmitic acid as the fatty acid, olive or jojoba oil chlorides are added as the fatty acid chloride. Artisans will readily recognize and understand the process of making the olive or jojoba oil chloride.

### EXAMPLE 3

Similarly, the procedure of EXAMPLE 1 is duplicated. However, rather than using palmitic acid as the fatty acid, one or more of the other fatty acids listed above is added as the fatty acid chloride. Artisans will readily recognize and understand the process of making the desired fatty acid chloride.

### EXAMPLE 4

Oils that have multiple fatty acids, each having different sized aliphatic chains may be used to create “time-release” stem cell secretion preparations. Shorter aliphatic chains are cleaved more quickly to expose the active site of the constituents of the stem cell secretion preparations, while the longer aliphatic chains are protected for longer. Thus, the net effect is an extended delivery time for the modified stem cell secretion preparations. Additionally, other growth factors, antioxidants, coenzymes, and biologically desirable or necessary molecules may be added to the stem cell secretions prior to adding fatty acids as supplements.

### EXAMPLE 5

According to embodiments, stem cell secretions include at least one of senescence, dermatopontin, ICAM1, IGF-3, Insulin-like growth factor binding protein 7, pleitrophin, vimentin, and Id-1 gene. Senescence induces a decrease of dermatopontin, ICAM-1, collagen 1 and 3, insulin-like growth factor binding protein 3, pleitrophin, HSP-27, SOD 1, and vimentin, and an increase of collagen 8, MMP-1 and MMP-3, heme oxygenase-1, insulin-like growth factor binding protein 7, and PGD2 synthase. Dermatopontin is an extracellular matrix protein with possible functions in cell-matrix interactions and matrix assembly. The protein is found in various tissues and many of its tyrosine residues are sulphated. Dermatopontin is postulated to modify the behavior of TGFbeta through interaction with decorin. ICAM1 (CD54) is typically expressed on endothelial cells and cells of the immune system. ICAM1 binds to integrins of type CD1 la/CD18, or CD1 lb/CD18. ICAM1 is also exploited by Rhinovirus as a receptor. Insulin-like growth factor binding protein 3. The protein forms a ternary complex with insulin-like growth factor acid-labile subunit (IGFALS) and either insulin-like growth factor (IGF) I or II. In this form, it circulates in the plasma, prolonging the half-life of IGFs and altering their interaction with cell surface receptors. Insulin-like growth factor binding protein 7: this protein binds insulin altering its interaction with cell surface receptors. Pleitrophin. Consistent with its role in promoting keratinocyte growth, PTN was upregulated during cutaneous wound healing in vivo. Vimentin. Cytoskeleton protein involved in cell shape changes during senescence. Id-1 gene. Generally, proliferating cells express multiple Id genes, whereas upon differentiation in many cell types, the expression of Id genes is down-regulated. The expressions of Id1, 1d2, and 1d3 are induced when the serum or growth factors are added to Go-arrested fibroblasts. In addition, abolishing Id proteins synthesis by antisense oligonucleotides blocks the quiescent fibroblasts into the cell cycle. These facts suggest that Id proteins play a role in the regulation of the cell cycle.

### EXAMPLE 6

According to embodiments, higher intensity ultrasound causes the secretion of healing factors from the stem cells into the stem cell secretion. Low-intensity pulsed ultrasound (LIPUS) was used for its distinct effects on biologic mineralization at intensities of <100 mW/cm². Intensity-dependent differences in the pattern of accelerated mineralization may be due to different alterations in regulation of collagenous matrix formation. We stimulated with 3 intensities of pulsed ultrasound at 1.5 mHz (30, 60 and 120 mW/cm²) on collagen post-translational modification and mineralization in human CK-15/19 skin stem cells.

In an aspect of the disclosure, a pharmaceutically acceptable cosmetic base may be enriched while adding the fatty acid-stem cell secretion additive and one or more stabilizers using one or more high frequency ultrasonification methods to provide one or more enriched cosmetic bases.

In another aspect of the disclosure, the enriched cosmetic base of may further comprise one or more modified glutathiones which may have important therapeutically additive properties as is known in the art. Furthermore, the enriched cosmetic base may comprise one or more modified carnosines which also may have important therapeutically additive properties as is known in the art. Without limiting the disclosure, in another embodiment the enriched cosmetic base may further comprise one or more modified glutathiones and one or more modified carnosines and the combination of the enriched cosmetic base, the one or more glutathiones and the one or more carnosines may be further lyophilized to dryness for future use. Of course, further stabilizers and ingredients suitable for compounding therapeutically active formulations may also be added.

According to embodiments the therapeutic efficacy of any of the enriched cosmetic bases described above may be tested for skin penetration by applying any of the enriched cosmetic bases to a portion of a skin tissue and observing the amount of penetration of the enriched cosmetic base into the skin tissue as is understood in the art. Furthermore, in another embodiment the therapeutic efficacy of any of the enriched cosmetic bases described above may be tested for bioavailability by applying the enriched cosmetic base to a portion of a skin tissue and observing the bioavailability of the enriched cosmetic base within the skin tissue.

In another embodiment, one or more compositions comprising one or more stem cell secretions as described above may further comprise a pharmaceutically acceptable carrier to make the composition effective for systemic administration. In an exemplary embodiment the efficacy of this composition may be tested by applying the enriched cosmetic base composition to a portion of a skin tissue and observing the bioavailability of the composition within the skin tissue.

In yet another aspect of the disclosure, a dosage form of one or more stem cell secretions, both fatty acid modified and unmodified, may further comprise one or more pharmaceutically acceptable carriers. In one or more embodiments the dosage form may be compounded and comprise one or more of the following: a tablet, a capsule, injection, a liquid, a powder, a lecithin granule, a liposome, an inhalant, a sublingual form, a suppository, an oral spray, dermal patches, creams, gels, lotions, masks, other topical applications, and the like as is understood in the art. Artisans will readily appreciate that fatty acid modified stem cell secretion taken orally are protected in the digestive tract and more readily absorbed to a greater extent than unprotected molecules.

In yet another aspect of the disclosure one or more enriched cosmetic bases may comprise one or more of the following: cocoa butter, aloe vera gel, aquafor, petroleum jelly, lecithin, almond oil, borage oil, canola oil, grape seed oil, jojoba oil, olive oil, soybean oil, sunflower oil, wheat germ oil, apricot kernel oil, carrot oil, coconut oil, hemp seed oil, flax seed oil, mango butter, evening primrose oil, black currant oil, avocado oil, microcrystalline wax, paraffin, petrolatum, petroleum jelly, ozokerite, montan wax, beeswax, at least one of lanolin and a derivative of lanolin, candelilla wax, ouricury wax, carnauba wax, Japan wax, cocoa butter, sugarcane wax, cork fiber wax, and the like as is understood in the art

In another aspect of the disclosure, the enriched compositions may be compounded to be systemically effective. Furthermore, in yet another aspect of the disclosure the enriched compositions described herein may comprise one or more pharmaceutically acceptable carriers comprising one or more of the following: a binding agent, a filler, a lubricant, a disintegrant, a wetting agent, a sugar, a starch, a cellulose and derivatives thereof, a stabilizer, a tableting agent, an antioxidant, a preservative, a colorant, a flavorant, and the like as is known in the art. In most compounding (as described herein) such pharmaceutically acceptable carriers may be also termed “excipients.”

With respect to therapeutic uses of one or more topically active dosage forms described above, enriched cosmetic base may be effective in rejuvenating a portion of an epithelial structure of a human skin, according to embodiments. Furthermore, without limiting the disclosure, in an aspect of the disclosure, in a therapeutic rejuvenation method, one or more of these topically active dosage forms may be applied topically for a period of from at least one to at least three times daily to a portion of an epithelial structure of a human skin.

According to embodiments, one or more topically active dosage forms as described above may be effective in restoring hair growth of a portion of an epithelial structure comprising one or more hair follicles. Furthermore, in one or more embodiments one or more topically active dosage forms may be applied to a portion of one or more epithelial structures of skin tissues and may be effective in reducing wrinkles, reducing cross-linking, controlling free radical deterioration, increasing collagen levels, increasing growth factors, reducing lipofuscin, reducing one or more inflammatory conditions, reducing benign epidermal proliferation or cancers, rejuvenating one or more traumatic conditions, and controlling dermal remodeling and scar formation to regrow skin tissue without abnormal appearance.

In another embodiment one or more enriched cosmetic bases compounded as topically active dosage forms (described above) may further comprise alpha-hydroxy lactic acid. Furthermore, these topically active dosage forms may be effective in reducing cohesive dermatomes after application to a portion of one or more skin tissues.

It will be readily appreciated that in another aspect of the disclosure, either one or more orally active or topically active dosage forms may protect against age-related decline, promote good health and slow premature aging by administering one or more enriched cosmetic bases (alone or in combination as a compounded composition) to one or more mammals, such as humans. In an aspect of the disclosure, these one or more orally active or topically active dosage may be administered according to one or more schedules as shown in the following: a single daily dose, in divided daily doses, in doses every other day, or in doses every three days and the like so as to insure proper effectiveness of the therapy, according to various embodiments.

It will be appreciated that the stem cell secretions of the present disclosure may be effective in improving heath and treating abnormal conditions in tissues other than skin. According to embodiments, one or more orally active or topically active dosage forms may comprise one or more of the following: a tablet, a capsule, an injection, a liquid, a powder, a lecithin granule, a liposome, an inhalant, a sublingual tablet, a suppository, an oral spray, a dermal patch, creams, gels, lotions, masks, other topical applications, and the like as is understood in the art. Furthermore, in another embodiment, one or more orally active or topically active dosage forms may be further configured with one or more pharmaceutically acceptable carriers of the following dosage forms: a tablet, a capsule, an injection, a liquid, a powder, a lecithin granule, a liposome, an inhalant, a sublingual tablet, a suppository, an oral spray, a dermal patch, creams, gels, lotions, masks, other topical applications, and the like as is understood in the art. These may contain fatty acid modified or unmodified stem cell secretions, or fatty acid modified or unmodified stem cell secretions and other supplements as described previously.

In further aspects of the disclosure, one or more body organs of a mammal may be rejuvenated by systemically administering one or more epidermal stem cell additives or compositions as described above. In an exemplary embodiment the one or more body organs may be the mammal's heart or kidney and the like, as is understood in the art and one or more compositions as described above may be to a mammal for the treatment of hepatic failure or to promote healing of fractures of a bone. Furthermore, in another aspect of the disclosure other benefits of administering one or more epidermal stem cell additives and compounds thereof (as described herein) may be to promote longevity in a mammal, quell inflammation in a mammal or reduce the risk of degenerative diseases in a mammal.

In an exemplary aspect of the disclosure, in a method of testing the therapeutic effectiveness of an epidermal stem cell additive a cosmetic base is provided; a composition comprising one or more stem cell additives is provided (as described herein); the cosmetic base and the epidermal stem cell additive are compounded to provide a cosmetic foundation; the compounded cosmetic foundation is applied to a portion of a surface of a skin tissue; the amount of penetration of the cosmetic foundation into the skin tissue is observed; and the bioavailability of the cosmetic foundation within the skin tissue is determined. According to embodiments at least a portion of the at least one stem cell additive is bound to at least one bio-marker configured to identify bioavailability of the cosmetic foundation within the skin tissue. In an aspect, the marker may comprise magnetite in the form of Minden beads and the like as is understood in the art.

In further aspects of the testing method described herein, the at least one stem cell secretion additive may be attached to at least one bio-marker, non-binding cells may be rinsed away from the at least one stem cell additive to leave a bound substantially bio-active residue; and the bound substantially bio-active residue may be enzymatically treated (as is known in the art) to provide an unbound substantially bioactive residue. Furthermore, each step according to this method may be repeated upon the bioactive residue until the purity of the at least one stem cell additive has reached a predetermined suitable level.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
harvesting stem cells;
culturing the stem cells;
separating the a stem cell conditioned media from the stem cells and other debris to produce purified stem cell secretions;
providing the stem cell secretions to be delivered to an animal.

2. The method of claim 1, further comprising lyophilizing or drying the stem cell secretions.

3. The method of claim 1, wherein the stem cell secretions are provided as an additive to a topically applied delivery vehicle.

4. The method of claim 3, wherein the delivery vehicle comprises at least one of a lotion, a skin cream, a topically applied patch, dermal injectable product, topically applied gel, tonic, cleansing agent, and a powder.

5. A product by the process of claim 1.

6. A product by the process of claim 3.

7. A method comprising:
harvesting stem cells;
culturing the stem cells;
removing a stem cell conditioned media having stem cell secretions;
adding a fatty acid chloride source to the aqueous solution at pH 11-14;
stirring;
purifying the resulting precipitate, the precipitate comprising at least fatty acid modified stem cell secretions.

8. The method of claim 1, wherein the stem cell are epidermal stem cells harvested from hair follicles.

9. The method of claim 1, wherein the reaction is performed at pH 12-13.

10. The method of claim 1, wherein the base source is NaOH.

11. The method of claim 1, wherein the aliphatic chain of the fatty acid chloride contains from 4-22 carbons.

12. The method of claim 1, wherein a plurality of fatty acids chlorides having varied aliphatic chain lengths comprise the fatty acid chloride source.

13. The method of claim 1, wherein the fatty acid chloride source comprises an oil.

14. The method of claim 13, wherein the oil is one of olive oil, almond oil, or jojoba oil.

15. The method of claim 1, wherein purification further comprises decanting the solution, washing the precipitate at least once, and drying the precipitate.

16. A product by the process of claim 1.

17. The product of claim 16, wherein the product is delivered to an animal.

18. The product of claim 17, wherein the product is delivered orally.

19. The product of claim 17, wherein the product is delivered topically.

20. The product of claim 19, wherein the product is added as an additive to a lotion.

21. A composition comprising:
stem cell secretions harvested from a conditioned stem cell media and separated from the stem cells from which the stem cell secretions are derived, wherein the stem cell secretions have at least one fatty acid chemically attached thereto.
